(19) United States
(12) Patent Application Publication
(10) Pub. No.: US 2024/0004516 A1
(43) Pub. Date: Jan. 4, 2024

(54) METHOD AND SYSTEM FOR CREATING A DATA MODEL

(71) Applicant: Prama, Inc., Louisville, KY (US)

(72) Inventor: Srinivasa Raju Eedarapalli, Louisville, KY (US)

(73) Assignee: Prama, Inc., (omitted)

(21) Appl. No.: 17/503,941

(22) Filed: Oct. 18, 2021

Related U.S. Application Data
(60) Provisional application No. 63/092,532, filed on Oct. 16, 2020.

(51) Int. Cl. G06F 3/0482; G06F 3/0484; G06F 16/901; G16H 40/20

(52) U.S. Cl. CPC G06F 3/0482; G06F 3/0484; G06F 16/9024; G16H 40/20

(57) ABSTRACT

The invention relates to method and system for creating a data model. The method includes receiving input to create process entity via first User Interface (UI). The process entity is configured to act as first-class citizen. The method further includes creating node corresponding to process entity to generate nodal network via second UI; dynamically receiving attributes associated with selected entity via the first UI; annotating node corresponding to the selected entity based on the attributes received via the second UI; and dynamically creating link between first and second nodes in the nodal network via the second UI. The second UI includes nodes. The link creation includes receiving selection of the first node; receiving selection of the second node; generating the link between the first node and the second node; and receiving direction input associated with the link; and defining direction of the link between the first and second nodes.

### TECHNICAL FIELD

Generally, the invention relates to data modeling. More specifically, the invention relates to a method and a system creating a data model.

### BACKGROUND

Domains are getting complex, because of large number of relationships that exist between the domain entities, and the evolving nature of those relationships. In modern day domains, it is important for data modeling tools to capture entities and their relationships along with the information and knowledge associated to these relationships without a loss in information, because any loss in entities and relationships and the related information associated with the entities and relationships is a loss in knowledge. This may further hamper the business domain.

Modeling data in a complex domain, e.g. a business domain, requires a lot of expertise and experience. The ability to visualize a domain's information needs at various levels of abstraction is hard. Even for experts, it may take several iterations to get it right, let alone complete. The current technologies lack a tool that provides for a structured yet natural approach to data modeling.

Current data modeling tools serve to model data structures supported by the Relational Database Management Systems (RDBMS). But, RDBMSs lack support for relationships in a more natural way.

Complex and connected nature of the modern day business models requires the data models and databases to support relationships as first class citizens. Using graph databases may be more appropriate in supporting RDBMS, but, the schema less (i.e. no predefined structure of database) nature of them may not be efficient to be used for every system or domain. For example, in Online Transaction Processing System (OLTP), its software development technologies work well with data that follows a well-defined schema, but may not work well with a schema less nature, because it may be very difficult to code against data with unknown structures. Further, ability to serialize and de-serialize the data into a language constructs in programming languages (ex. POCO/POJO classes in Java/C #) needs to be written using this data, may only be done when the data has pre-defined structure/schema.

Thus, there is a need to address the above problems in the current systems for modelling and structuring data models in domains.

### SUMMARY

In one embodiment, a method for creating a data model is disclosed. The method may include receiving, via a first User-Interface (UI), an input to create a process entity. It should be noted that the process entity may be configured to act as first-class citizen. The method may include creating, via a second UI, a node corresponding to the process entity to further generate a nodal network. The second UI may include a plurality of nodes. The method may further include dynamically receiving, via the first UI, one or more attributes associated with a selected entity. The method may further include annotating, via the second UI, a node corresponding to the selected entity based on the one or more attributes received. The method may further include dynamically creating, via the second UI, a link between a first node and a second node in the nodal network. Link creation may further include receiving a selection of the first node, receiving a selection of the second node, generating the link between the first node and the second node, receiving a direction input associated with the link, via the second UI, and defining the direction of the link between the first node and the second node. The link may be configured to act as a first-class citizen.

In another embodiment, a system for creating a data model is disclosed. The system may include a processor and a memory communicatively coupled to the processor. The memory may store processor-executable instructions, which, on execution, may cause the processor to receive, via a first User-Interface (UI), an input to create a process entity. It should be noted that the process entity may be configured to act as first-class citizen. The processor-executable instructions, on execution, may further cause the processor to create, via a second UI, a node corresponding to the process entity to further generate a nodal network. The second UI may include a plurality of nodes. The processor-executable instructions, on execution, may further cause the processor to dynamically receive, via the first UI, one or more attributes associated with a selected entity. The processor-executable instructions, on execution, may further cause the processor to annotate, via the second UI, a node corresponding to the selected entity based on the one or more attributes received. The processor-executable instructions, on execution, may further cause the processor to dynamically create, via the second UI, a link between a first node and a second node in the nodal network. Link creation may further include receive a selection of the first node, receive a selection of the second node, generate the link between the first node and the second node, receive a direction input associated with the link, via the second UI, and define the direction of the link between the first node and the second node. The link may be configured to act as a first-class citizen.

In yet another embodiment, a non-transitory computer-readable medium storing computer-executable instruction for creating a data model is disclosed. The stored instructions, when executed by a processor, may cause the processor to perform operations including receiving an input to create a process entity via a first User-Interface (UI). It should be noted that the process entity may be configured to act as first-class citizen. The operations may further include creating a node corresponding to the process entity to further generate a nodal network via a second UI. The second UI may include a plurality of nodes. The operations may further include dynamically receiving one or more attributes associated with a selected entity via the first UI. The operations may further include annotating a node corresponding to the selected entity based on the one or more attributes received, via the second UI. The operations may further include dynamically creating a link between a first node and a second node in the nodal network, via the second UI. Link creation may further include receiving a selection of the first node, receiving a selection of the second node, generating the link between the first node and the second node, receiving a direction input associated with the link, via the second UI, and defining the direction of the link between the first node and the second node. The link may be configured to act as a first-class citizen.

It is to be understood that both the foregoing general description and the following detailed description are exemplary and explanatory only and are not restrictive of the invention, as claimed.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a block diagram of an exemplary system for creating a data model, in accordance with some embodiments of the present disclosure.

FIG. 2 illustrates a functional block diagram of various modules within memory of a creation device, in accordance with some embodiments of the present disclosure.

FIG. 3 illustrates a block diagram of a system for creating and modifying the multi-dimensional data model, in accordance with another embodiment of the present disclosure.

FIG. 4 illustrates a flowchart of a method for creating a data model, in accordance with some embodiments of the present disclosure.

FIG. 5 illustrates a flowchart of a method for creating and dynamically modifying the multi-dimensional data model, in accordance with some embodiments of the present disclosure.

FIG. 6 illustrates a User Interface (UI) representing creation of an entity, in accordance with some embodiments of the present disclosure.

FIG. 7 illustrates a User Interface (UI) representing association of an entity with respective attributes, in accordance with some embodiments of the present disclosure.

FIG. 8 illustrates a User Interface (UI) that represents entity attributes provided to an entity, in accordance with some embodiments of the present disclosure.

FIGS. 9A-9B illustrate User Interfaces (UIs) for inputting information related to the entity attributes, in accordance with some embodiments of the present disclosure.

FIGS. 10A-10D illustrate User Interfaces (UIs) for adding, creating, and representing another entity, in accordance with some embodiments of the present disclosure.

FIGS. 11A-11B illustrate User Interfaces (UIs) employed to create relationship types between data entities, in accordance with some embodiments of the present disclosure.

FIG. 11C illustrates a User Interface (UI) employed to provide and associate attributes to relationship types, in accordance with some embodiments of the present disclosure.

FIG. 11D illustrates a User Interface (UI) employed for editing, in accordance with some embodiments of the present disclosure.

FIGS. 12A-12B illustrate a directed graph-based User Interface (UI) that represent a multi-dimensional data model, in accordance with some embodiments of the present disclosure.

FIGS. 13A and 13B illustrate user interfaces where the data captured in the multi-dimensional data model may be used as sample data for various applications, in accordance with some embodiments of the present disclosure.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the present invention is not intended to be limited to the embodiments shown, but is to be accorded the widest scope consistent with the principles and features disclosed herein.

While the invention is described in terms of particular examples and illustrative figures, those of ordinary skill in the art will recognize that the invention is not limited to the examples or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic (The term “logic” herein refers to fixed hardware, programmable logic and/or an appropriate combination thereof, as would be recognized by one skilled in the art to carry out the recited functions.) Software and firmware can be stored on computer-readable storage media. Some other processes can be implemented using analog circuitry, as is well known to one of ordinary skill in the art. Additionally, memory or other storage, as well as communication components, may be employed in embodiments of the invention.

The present disclosure provides a system and a related method for designing and modeling of a data model, and more particularly to a method and a system for data modeling and data visualization for a relational database, such as Relational Database Management Systems (RDBMS). The present system may provide a graph-based relational data model for supporting a relational database. Further, the present system may provide an interactive and easy way to create and model a data model via an interactive user interface.

The system includes a model creation device that helps in modelling data structures by implementing use cases or examples approach, as and when data comes, in any shape or form, and over time. The system obtains data from one or more best examples that may be provided in a domain by domain experts and employs this data from the examples to create a data model from scratch.

The system develops graph based data model by layering complex schema on top of Graph Data Model/Database.

Further, the system model data structures that may efficiently capture attributes, information and knowledge in data entities, as well as in the relationships between the data entities.

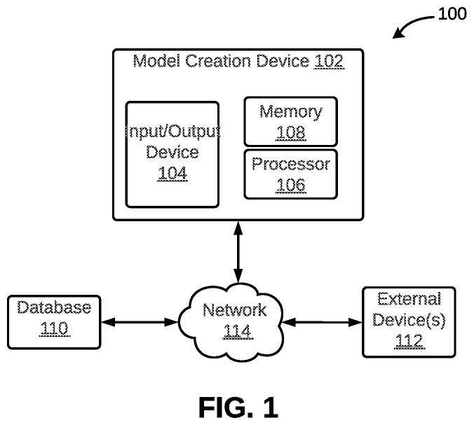

Referring to FIG. 1, a block diagram of system 100 for creating a data model is illustrated, in accordance with some embodiments of the present disclosure. The system 100 may create and manipulating a multi-dimensional relational data model for supporting a database. In an embodiment, the multi-dimensional relational data model may support a RDBMS. The multi-dimensional model may represent a relationship database model that may include multiple relationships between multiple entities. By creating and modeling the multi-dimensional data model, the relationship database model for supporting RDBMS, in turn, may be created and modeled.

The system 100 may include a model creation device 102 with processing capabilities for creating, manipulating/modeling, and viewing the multi-dimensional data model. Manipulating may include modifying the data model by adding, deleting, updating, or changing any attribute of the model. Examples of the model creation device 102 may include, but are not limited to a server, a desktop, a laptop, a notebook, a net book, a tablet, a smartphone, or a mobile phone. The model creation device 102 may include an input/output (I/O) device 104 that may be responsible for capturing information and data, for processing, and in turn displaying a processed output. By way of an example, the I/O device 104 may be used to display results of analysis performed by the model creation device 102, to the user. By way of another example, the I/O device 104 may be used by the user to provide inputs to the model creation device 102. Thus, for example, in some embodiments, the model creation device 102 may ingest information such as an input to create process entity, and attributes associated with the selected entity for annotating nodes via a user interface of the I/O device 104 (not shown in FIG. 1). Further, for example, in some embodiments, the model creation device 102 may render results to the user/administrator via the user interface. The input/output device 104 may include, but is not limited to, a keyboard, a mouse, a microphone, a scanner, a joystick, a monitor, a digital screen, and a printer.

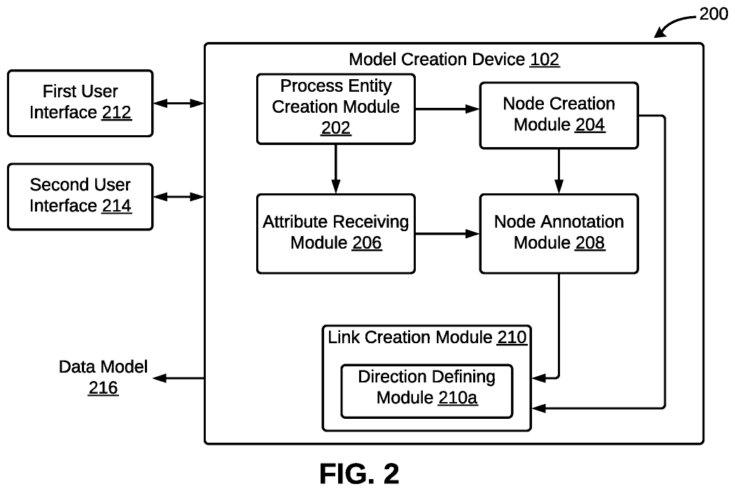

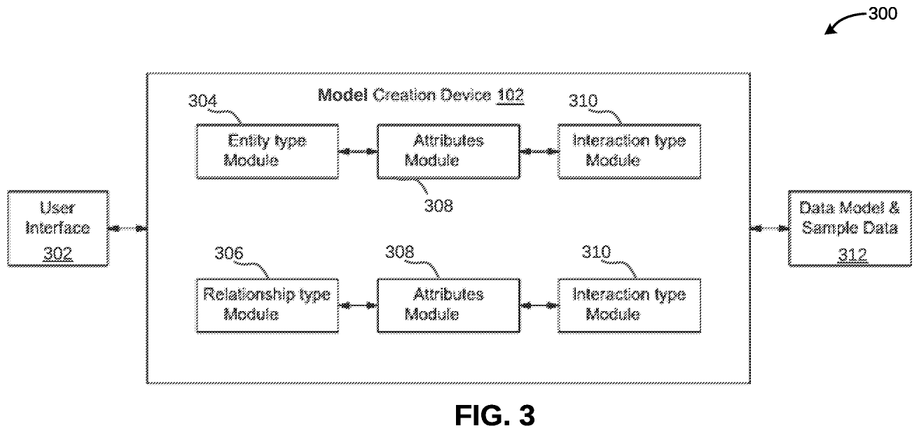

The model creation device 102 may further include a processor 106, which may be communicatively coupled to a memory 108. The memory 108 may store processor instructions, which when executed by the processor 106 may cause the processor 106 to the multi-dimensional data model in turn to modify the relationship database model for supporting RDBMS. This is further explained, in detail in conjunction with FIGS. 2 and 3. The processor 106 in conjunction with the memory 108 may perform various functions including creating a node, generating a nodal network, annotating selected entity, creating links, and the like.

The memory 108 may store various data (for example, plurality of nodes, attributes associated with nodes, link data, and the like) that may be captured, processed, and/or required by the model creation device 102. The memory 108 may be a non-volatile memory or a volatile memory. Examples of non-volatile memory, may include, but are not limited to a flash memory, a Read Only Memory (ROM), a Programmable ROM (PROM), Erasable PROM (EPROM), and Electrically EPROM (EEPROM) memory. Examples of volatile memory may include, but are not limited to, Dynamic Random-Access Memory (DRAM), and Static Random-Access memory (SRAM).

Moreover, the model creation device 102 may be communicatively coupled to database 110 storing information related, including but not limited to, one or more users of the software system/application and/or of the business organization, one or more information related to business processes in the business organization or information related to the business organization. The database 110 may further include information related to the relationship database model for the RDBMS. It may be apparent to a person skilled in the art that the database 110 may include any type of information required by the system 100 and the model creation device 102, without deviating from the meaning and scope of the present disclosure. The database 110 may be updated periodically with a new set of parameters corresponding to the information stored in it.

Additionally, the model creation device 102 may be communicatively coupled to an external device 112 via network 114 for sending and receiving various data. Examples of the external device 112 may include, but is not limited to, a remote server, digital devices, and a computer system.

The network 114 may correspond to a communication network. The communication network 114, for example, may be any wired or wireless communication network and the examples may include, but may be not limited to, the Internet, Wireless Local Area Network (WLAN), Wi-Fi, Long Term Evolution (LTE), Worldwide Interoperability for Microwave Access (WiMAX), and General Packet Radio Service (GPRS). In some embodiments, the database 110 may be directly coupled to the model creation device 102. In some other embodiments, the database 110 may be coupled to the model creation device 102 via the network 114.

Referring now to FIG. 2, a functional block diagram of various modules 202-210 within memory of the model creation device 102 is illustrated, in accordance with some embodiments of the present disclosure. The model creation device 102 may include a process entity creation module 202, a node creation module 204, an attribute receiving module 206, a node annotation module 208, and a link creation module 210, to perform various functions, via a first User Interface (UI) 212 and a second UI 214, while creating a data model 216. Further, in one embodiment, a user may provide inputs to the modules 202-210 via the first user interface 212. In some other embodiments, the user may be able access the nodes of the network model via the second user interface 214. FIG. 2 is explained in conjunction with FIG. 1.

The process entity creation module 202 may be configured to receive an input to create a process entity via the first UI 212. The process entity may be configured to act as first-class citizen. The process entity creation module 202 may be communicatively coupled to the node creation module 204 and the attribute receiving module 206, as illustrated in FIG. 2.

The node creation module 204 may be configured to create a node corresponding to the process entity, via the second UI 214. The node creation may further lead to generate a nodal network. It should be noted that the second UI may include a plurality of nodes corresponding to different process entities. The node creation module 204 may be operatively coupled to the node annotation module 208 and the link creation module 210. The attribute receiving module 206 may receive one or more attributes associated with a selected entity via the first UI 212. The attribute receiving module 206 may transmit information corresponding to the received one or more attributes to the operatively coupled node annotation module 208.

The node annotation module 208 may be configured for annotating a node corresponding to the selected entity based on the one or more attributes received, via the second UI 214. The node annotation may be further coupled to the link creation module 210 to transmit the annotated node.

Further, the link creation module 210 may dynamically create a link between a first node and a second node, in the nodal network, via the second UI 214. In one embodiment the link creation module 210 may receive a selection of the first node via the second UI 214. In one embodiment, the link creation module 210 may receive a selection of the second node via the second UI 214. In one embodiment, the link between the first node and the second node may be generated by the link creation module 210. Further, the link creation module 210 may include a direction defining module 210a. The direction defining module 210a may be configured to receive a direction input associated with the link via the second UI 214 and subsequently define the direction of the link between the first node and the second node. It should be noted that the link may be configured to act as a first-class citizen.

Further, the model creation device 102 may also include a rendering module (not shown in FIG. 2). The rendering module may be configured to render the nodal network in a vector format via the second UI 214. The nodal network may be configured to be zoomed-in and zoomed-out This may be further explained in conjunction with FIGS. 3-13.

Additionally, the model creation device 102 may include a module that may define one or more attributes to the link between the first node and the second node in the nodal network. Also, a classification module (not shown in FIG. 2) may be utilized by the model creation device 102 to classify the link between the first node and the second node in the nodal network in one or more categories based on the one or more attributes.

Further, in some embodiments, the model creation device 102 may include an attribute selection module (not shown in FIG. 2). The attribute selection module may be configured to select one or more aesthetic attributes for each node in the nodal network. The one or more aesthetic attributes may include, but not limited to, a shape, a size, and a colour.

The model creation device 102, in some embodiments, may receive at least one of a selection of a portion of the one the nodal network, via the second UI 214, a selection of an attribute from the one or more attributes associated with a node, or a selection of an attribute from the one or more attributes associated with a link. Further the rendering module may render a portion of the nodal network based on one of the selection of the portion of the one the nodal network, the selection of the attribute from the one or more attributes associated with the node, or the selection of the attribute from the one or more attributes associated with the link.

Referring now to FIG. 3, a block diagram of a system 300 for creating and modifying the multi-dimensional data model 312 for supporting a relational database is illustrated, in accordance with some embodiments of the present disclosure. FIG. 3 is explained in conjunction with FIGS. 1-2. In an embodiment, the multi-dimensional relational data model 312 may be for supporting a RDBMS. As explained in conjunction to FIG. 1, the model creation device 102, may act as an interactive interface for providing a complete view of the multi-dimensional data model 312. The system 300 may include various modules within the memory 108 configured to work together.

The model creation device 102 may interact with a user via a user interface (UI) 302. In an embodiment, the user interface (UI) 302 allows the user to interact with the system 100 and input any data content in the system 100. In an embodiment, the UI 302 may include an input and output user interface, individually or in combination. The output user interface may allow the system 100 to output any content to the user. The input user interface may include and is not limited to voice-enabled user interface, a touch-sensitive screen, non-touch sensitive screen, touch keypad or a non-touch keypad, a keyboard, a mouse, a stylus, an image/video capturing device, such as camera, one or more push buttons, soft keys and the like. The output user interface may include and is not limited to a display screen, a speaker, and the like.

Further, the model creation device 102 may include one or more operating modules to create and modify the data model 312, in the present disclosure. The one or more modules may include an entity type module 304 and a relationship type module 306. The entity type module 304 and the relationship type module 306 may operatively communicate and function along with one single attributes module 308. In an embodiment, the entity type module 304 and the relationship type module 306 may respectively communicate with their respective attributes module 308. Further, the entity type module 304 and the relationship type module 306, in operative communication with the attributes module 308, may operatively communicate and function along with an interaction type module 310. The entity type module 304, the relationship type module 306, the attributes module 308 and the interaction type module 310 may function together to create and model the multi-dimensional data model 312, according to an embodiment of the present disclosure.

It may be apparent to a person ordinary skilled in the art that the model creation device 102 may include operating modules other than the entity type module 304 and the relationship type module 306, the attributes module 308 and the interaction type module 310, for creating and modelling the data model 312, without deviating from the meaning and scope of the present invention.

According to an embodiment, a user of the present system 100 may utilize the UI 302 and input information related to the data model 312, in order to create the data model 312. For example, the model creation 302 employs the entity type module 304 to obtain information related to one or more entities to be included in the data model 312. The entity type module 304 functions with the attributes model 308 to obtain one or more attributes related to an entity. An entity may refer to any singular, identifiable and separate object. It may include and is not limited to individuals, organizations, systems, bits of data or even distinct system components that are considered significant in and of themselves. For example, in a medical domain, an entity may include a patient, a doctor, a medical expert, a medical specialization, a medical condition, a hospital, a medical visit, and the like. [036]Further, as mentioned above, the entity type module 304 functions with the attributes model 308 to obtain one or more attributes related to an entity. Thus, the model creation device 102 may obtain one or more attributes or properties defining the entity which is created. Attributes may include and is not limited any information, data, capabilities and/or functions related to the entity. For example, in a medical domain attributes related to an entity ‘patient’ may include ‘first name’, ‘last name’, ‘address’, ‘medical condition’, and the like.

Furthermore, the system 100 utilizing the model creation device 102 may be able to efficiently capture information for the attributes and the related entities. Thus, the system 100 may capture knowledge related to the attributes and the entities, without loss of fidelity. To capture information in the attributes and the related entities, the interaction type module 310 may receive, via the UI 302, use case or example data corresponding to the entities, based on a real time activity performed in the use case or example. Any kind of user interaction with the system 100 may take place through the UI 302.

If the user interaction includes adding, updating, or manipulating the entities in the multi-dimensional data model 312, then the same is updated in the data model 312, using the interaction type module 310. The interaction type module 310 may, also, receive use case data related to a use case, as and when such data comes with time in any shape or form, thus the interaction type module 310 may also be utilized in updating the multi-dimensional data model 312 for supporting the RDBMS in real time, as and when data comes.

Furthermore, the model creation device 102 may utilize the entity module 304, along with the attributes module 308 and interaction type module 310, to add, edit or modify the new and existing entities and their new and existing attributes too and the information contained in those attributes.

Utilizing the model creation device 102, the system 100 may also create relationships between the entities which are created as explained above. The relationships module 306 may receive, via the UI 302, an input related to interconnection relationships between the individual entities. Such input may represent data/attributes including and not limited to how, what and may be why each of the individual entities are connected with each other. The relationships between the entities which may be created and modelled, using the relationships module 306, may also receive one or more relationship attributes or properties, employing the attributes module 308 along with the relationships module 306. The relationship attributes or properties may define the relationships between the entities. Relationship attributes may include and is not limited any information, data, capabilities and/or functions related to the relationship. In an embodiment, the relationships may be unidirectional or bi-directional. In an embodiment, single entity can be connected to multiple entities via multiple relationships.

Furthermore, in an embodiment, the system 100 utilizing the model creation device 102 may be able to efficiently capture information for the attributes and the related relationships. Thus, the system 100 may capture knowledge related to the attributes and the relationships between the entities, without loss of fidelity. To capture information in the attributes and the related relationships between the entities, the interaction type module 310 may receive, via the UI 302, use case or example data, corresponding to the relationships between the entities, based on a real time activity performed in the use case or example. Any kind of user interaction with the system 100 may take place through the UI 302. If the user interaction includes adding, updating, or manipulating the relationships between the entities in the multi-dimensional data model 312, then the same is updated in the data model 312, using the interaction type module 310. The interaction type module 310 may, also, receive use case data related to a use case, as and when such data related to relationships between the entities comes with time in any shape or form, thus the interaction type module 310 may also be utilized in updating the multi-dimensional data model 312 in real time, as and when such use case data comes.

For example, in a medical domain, a relationship between an entity ‘patient’ and ‘visit’ may be created and provided with attributes such as ‘made a’. Also, a relationship between an entity ‘doctor’ and ‘specialization’ may be created and provided with attributes such as ‘specializes in’.

Furthermore, the model creation device 102 may utilize the relationships module 306, along with the attributes module 308 and interaction type module 310, to add, edit or modify the new and existing relationships and their new and existing attributes too and the information contained in those attributes.

Thus, the model creation device 102 of the system 100 may create a data model 312 using at least the entity type module 304 and the relationships module 306, along with the attributes module 308 and interaction type module 310, individually or in any combination. Also, because the system 100 is example approach driven and utilizes use case or example data to create the multi-dimensional data model 312, the system 100 automatically creates sample data, during the process of data modeling itself, and this sample data may help the users in various applications, such as helps in using the data as mock data to build rest of software application.

It should be noted that all such aforementioned modules 302-310 may be represented as a single module or any combination of different modules. Further, as will be appreciated by those skilled in the art, each of the modules 302-310 may reside, in whole or in parts, on one device or multiple devices in communication with each other. In some embodiments, each of the modules 302-310 may be implemented as dedicated hardware circuit comprising custom application-specific integrated circuit (ASIC) or gate arrays, off-the-shelf semiconductors such as logic chips, transistors, or other discrete components.

Each of the modules 302-310 may also be implemented in a programmable hardware device such as a field programmable gate array (FPGA), programmable array logic, programmable logic device, and so forth. Alternatively, each of the modules 302-310 may be implemented in software for execution by various types of processors (e.g., processor 106). An identified module of executable code may, for instance, include one or more physical or logical blocks of computer instructions which may, for instance, be organized as an object, procedure, function, or other construct. Nevertheless, the executables of an identified module or component need not be physically located together but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

Further, it should be noted that, the model creation device 102 may be implemented in programmable hardware devices such as programmable gate arrays, programmable array logic, programmable logic devices, or the like. Alternatively, the system 100 and associated model creation device 102 may be implemented in software for execution by various types of processors. An identified engine/module of executable code may, for instance, include one or more physical or logical blocks of computer instructions which may, for instance, be organized as a component, module, procedure, function, or other construct. Nevertheless, the executables of an identified engine/module need not be physically located together but may include disparate instructions stored in different locations which, when joined logically together, comprise the identified engine/module and achieve the stated purpose of the identified engine/module. Indeed, an engine or a module of executable code may be a single instruction, or many instructions, and may even be distributed over several different code segments, among different applications, and across several memory devices.

As will be appreciated by one skilled in the art, a variety of processes may be employed for creating a data model. For example, the exemplary system 100 and associated model creation device 102 may create the data model, by the process discussed herein. In particular, as will be appreciated by those of ordinary skill in the art, control logic and/or automated routines for performing the techniques and steps described herein implemented by the system 100 and the associated model creation device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some or all the processes described herein may be included in the one or more processors on the system 100.

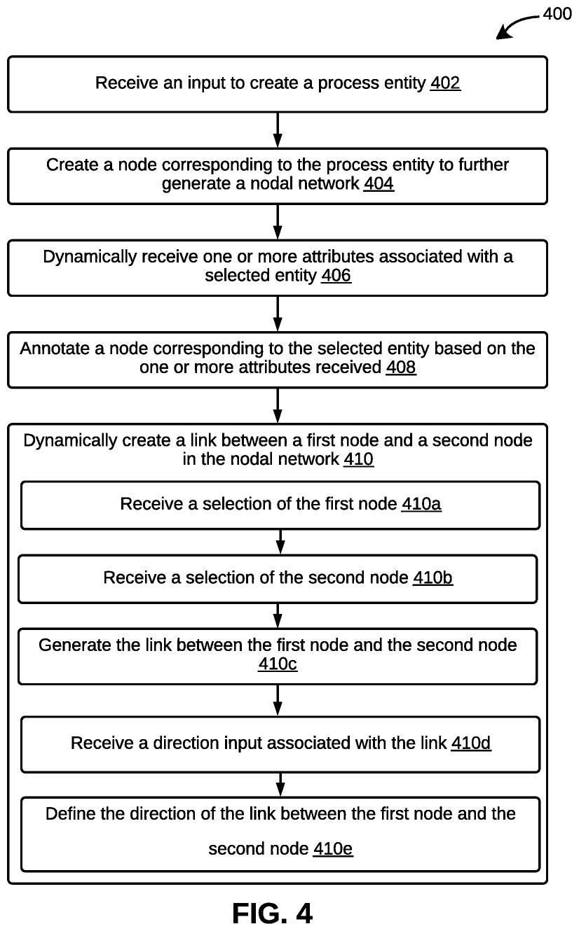

Referring now to FIG. 4, a method 400 for creating a data model is depicted via a flow diagram, in accordance with some embodiments of the present disclosure. Each step of the method 400 may be performed by the model creation device 102. FIG. 4 is explained in conjunction with FIGS. 1-3.

At step 402, an input may be received to create a process entity. The input entity may be received via a first User-Interface (same as the first user interface 212) by a process entity creation module (same as the process entity creation module 202). It should be noted that the process entity may be configured to act as first-class citizen.

At step 404, a node may be created corresponding to the process entity. The node may be created via a second User-Interface (same as the second user interface 214) using a node creation module (analogous to the node creation module 204). Further, the second User-Interface may include a plurality of nodes. It may be noted that the nodes, corresponding to each of received process entities, may be created to generate a nodal network. The nodal network may be rendered in a vector format via the second User-Interface, in accordance with some embodiments. And, the nodal network may be configured to be zoomed-in and zoomed-out.

Thereafter, at step 406, one or more attributes associated with a selected entity may be received dynamically via the first user interface. Further, at step 408, a node corresponding to the selected entity may be annotated via the second user interface. The annotation may be performed based on the one or more attributes received. And, a node annotation module (analogous to the node annotation module 208) may be utilized for performing the step 408.

At step 410, a link between a first node and a second node in the nodal network may be created via the second user interface, using a link creation module (similar to the link creation module 210). In some embodiments, at step 410a a selection of the first node may be received via the second user interface. In some embodiments, at step 410b, a selection of the second node may be received via the second user interface. Additionally, in some embodiments, the link between the first node and the second node may be generated, at step 410c. Further, in some embodiments, at step 410d, a direction input associated with the link may be received via the second user interface. In some embodiments, the direction of the link between the first node and the second node may be defined, using a direction defining module (same as the direction defining module 210a). It should be noted that the link may be configured to act as a first-class citizen.

Further, in some embodiments, one or more attributes to the link between the first node and the second node in the nodal network may be defined. The link between the first node and the second node in the nodal network may be classified in one or more categories based on the one or more attributes. In one embodiment, one or more aesthetic attributes may be selected for each node in the nodal network. The one or more aesthetic attributes may include at least one of a shape, a size, and a colour.

Further, in some embodiments, a selection of a portion of the one the nodal network may be received via the second user interface. Alternatively, in other embodiments, a selection of an attribute from the one or more attributes associated with a node may be received, or a selection of an attribute from the one or more attributes associated with a link may be received. Thereafter, a portion of the nodal network based on one of the selection of the portion of the one the nodal network, the selection of the attribute from the one or more attributes associated with the node, or the selection of the attribute from the one or more attributes associated with the link may be rendered, in accordance with some embodiments.

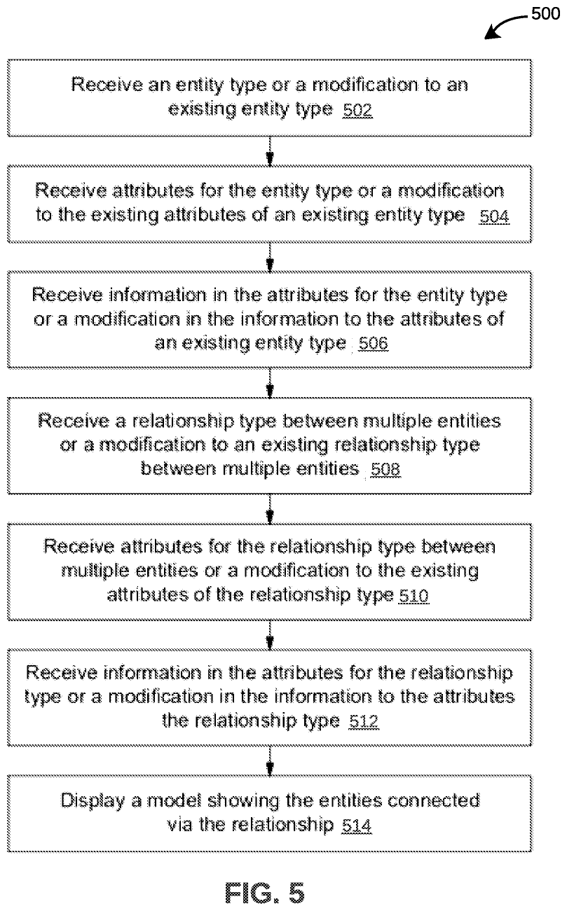

Referring now to FIG. 5, a method 500 for creating and dynamically modifying the multi-dimensional data model for a relational database is depicted via a flow diagram, in accordance with some embodiments of the present disclosure. Each step of the method 500 may be performed by the model creation device 102. FIG. 5 is explained in conjunction with FIGS. 1-4. Further, the method 500 may or may not follow a step flow as described by steps 502-514 in the flowchart in FIG. 5.

The method 500 may include capturing information or data in use cases for creating and modeling a multi-dimensional relationship data model in a domain, by receiving in use cases information or data, as and when it comes, in any shape and form, over time.

The method 500 may include receiving inputs to create one or more entities, via the UI 302 using the entity type module 304, according to an embodiment.

At step 502, entity information may be received using the entity type module 304, by the model creation device 102 via the UI 302. Alternatively, the entity type module 304 may receive a modification in an existing entity via the UI 302. After receiving entity information, the model creation device 102 may create one or more entities. Further, at step 504, one or more entity attributes defining the entities may be received using the entity type module 304 along with attribute module 308, by the model creation device 102 via the UI 302. Alternatively, the entity type module 304 may also receive a modification in existing attributes of the entity via the UI 302. After receiving entity attributes, the model creation device 102 may associate the one or more attributes with the respective entities.

Furthermore, at step 506, the model creation device 102 may capture and receive information to be contained in the attributes related to the entities, using the interaction type module 510, along with the entity type module 304 and the attribute module 308. Thus, at step 506 the system 100 captures knowledge related to the information contained in the attributes of the entities, and present them in the data model. Alternatively, the interaction type module 310 may also receive modifications in the existing information contained in the attributes related to the entities via the UI 302. After receiving the information, the model creation device 102 may associate the information with the one or more attributes respective to the entities.

Furthermore, the method 500 may include receiving inputs to create one or more relationships between the entities, via the UI 302 using the relationship type module 306, according to an embodiment. At step 508, relationship information may be received using the relationship type module 306, by the model creation device 102 via the UI 302. Alternatively, the relationship type module 304 may also receive a modification in an existing relationship via the UI 302. After receiving relationship information, the model creation device 102 may create relationships between the entities. Further, at step 510, one or more relationship attributes defining the relationship between the entities may be received using the relationship type module 306 along with attribute module 308, by the model creation device 102 via the UI 302. Alternatively, the relationship type module 306 may also receive a modification in existing attributes of the relationship between the entities via the UI 302. After receiving relationship attributes, the model creation device 102 may associate the one or more relationship attributes with the respective relationships between the entities.

Furthermore, at step 512, the model creation device 102 may capture and receive information to be contained in the attributes related to the relationships between the entities, using the interaction type module 310, along with the relationship type module 306 and the attribute module 308. Thus, at step 512 the system 100 captures knowledge related to the information contained in the attributes of the relationships between the entities and present them in the data model. Alternatively, the interaction type module 310 may also receive modifications in the existing information contained in the attributes related to the relationship between the entities via the UI 302. After receiving the information, the model creation device 102 may associate the relationship information with the one or more relationship attributes respective to the relationships between the entities.

The model creation device 102 of the system 100 may repeat the steps 502-512 as many times as required, and as many times as use cases data come. Executing the steps 502-512, the model creation device 102 creates the multi-dimensional data model representing a relationship data model. At step 514, the system 100 may display the multi-dimensional data model representing the relationship data model at the UI 302. Additionally, at the same step 514, the system 100 may also create sample data, during the process of data modeling itself (steps 502-512), and this sample data may also help the users in various applications, such as in using the data as mock data to build rest of software application.

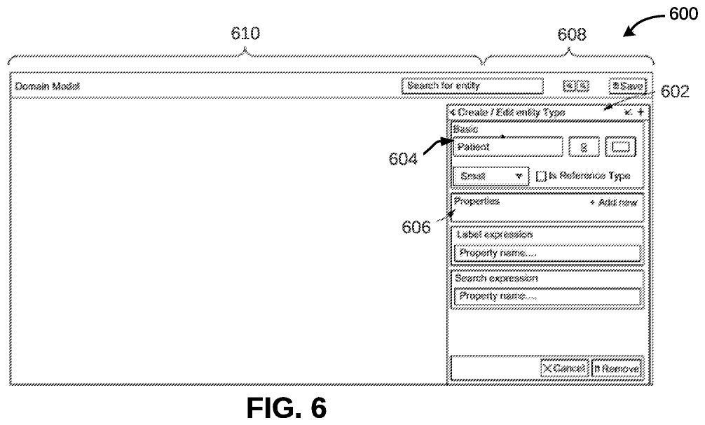

The multi-dimensional data model created/updated employing the FIGS. 1-5 above, is shown in the FIGS. 6-13.

A directed graph-based user interface that represents the multi-dimensional data model is further explained in detail in conjunction with FIGS. 6-13. FIGS. 6-13 illustrate directed graph-based user interfaces that show creation and modeling of the multi-dimensional data model representing the relationship data model. The directed graph-based user interfaces (UI) as shown in the FIGS. 6-13 may be understood and may function as the UI 302 of the system 300, as explained in the FIGS. 1-5 above. It should be understood that the present disclosure may not restrict to the directed graph-based user interfaces (UI) as shown in the FIGS. 6-13 for creation and modeling of the multi-dimensional data model, and may include and implement other user interfaces, without deviating from the meaning and scope of the present disclosure.

Also, the directed graph-based user interfaces (UI) as shown in the FIGS. 6-13 are explained by taking an exemplary situation of a medical domain. It should be understood that the present disclosure does not restrict to this exemplary situation of a medical domain and may be equally applicable for different domains, without deviating from the meaning and scope of the present disclosure. The directed graph-based user interfaces (UI) may function along with the model creation device 102 including one or more of its operating modules, as explained in the FIGS. 1-5.

Also, in an embodiment, the directed graph-based user interfaces (UI) of the FIGS. 6-13 may include a section that may be referred to as “creation section”, which may include one or more tabs for creating, modifying and modeling the data model. The directed graph-based user interfaces (UI) of the FIGS. 6-13 may also include a section that may be referred to as “model representation section” that may illustrate the multi-dimensional data model, as and when it is being created using one more tabs in the creation section.

Referring now to FIG. 6, a user interface (UI) 600 representing creation of an entity is illustrated, in accordance with an embodiment of the present invention. The UI 600 shows creation of an entity ‘patient’. A tab ‘602’ may be selected to create a new entity. A tab ‘604’ may be used to define a type of the entity, such as the ‘patient’. Additionally, a tab ‘606’ may be utilized to add/create attributes defining the entity ‘patient’. The tabs 602, 604, and 606 may be included in the right side of the UI 600, which may be the “creation section”.

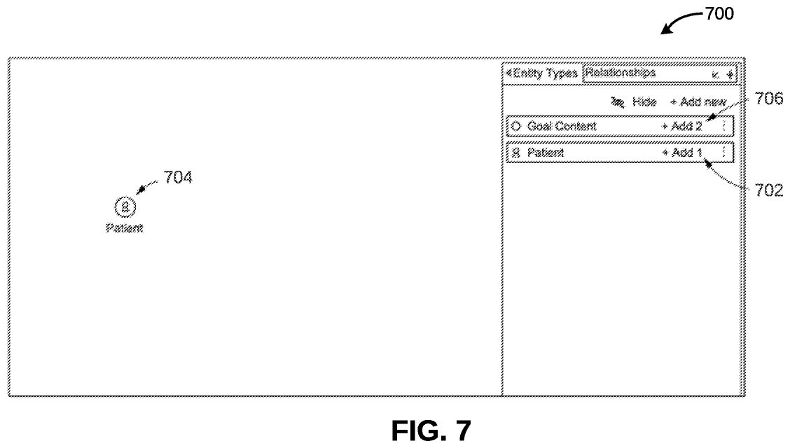

Referring now to FIG. 7, a UI 700 representing association of an entity with respective attributes is illustrated, in accordance with an embodiment of the present invention. Following, the UI 600 shown in the FIG. 6, the UI700 shows that the entity ‘patient’ is created associated with the respective attributes. On right side of the UI 700, an entity ‘patient’ 702 is shown in the creation section of the UI 700, whereas soon after the entity ‘patient’ is created in the FIG. 7, the UI 700 shows the created entity ‘patient’ as a node entity 704 to be included in the multi-dimensional relational data model, in the model representation section of the UI 700. Using the tab 706, more different entities may be created.

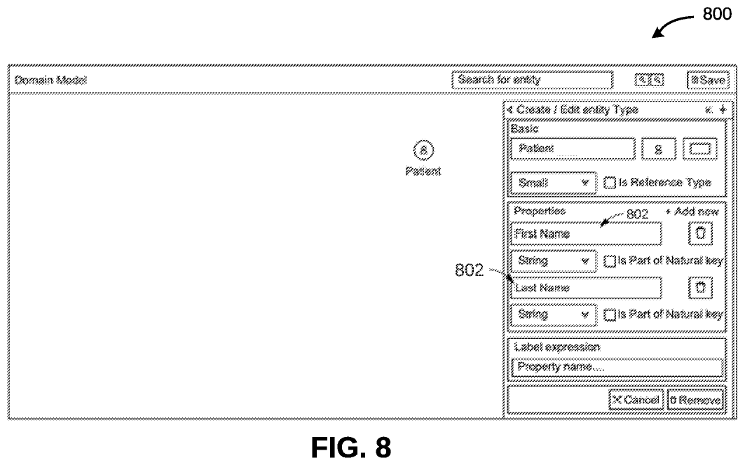

Referring now to FIG. 8, a UI 800 that represents entity attributes provided to an entity is illustrated, in accordance with an embodiment of the present invention. After creating and representing the entity ‘patient’ 702 in the FIGS. 6-7, the UI800 shows providing one or more entity attributes to the entity ‘patient’, that further defines the entity ‘patient’ 702. A tab 802 may be employed to create and define the entity attributes for the entity ‘patient’ 702, such as a ‘first name’ and a ‘last name’. Also, modifications to these attributes, such as modifying or adding or deleting attributes for the entity ‘patient’ may be done, using the tabs in the creation section.

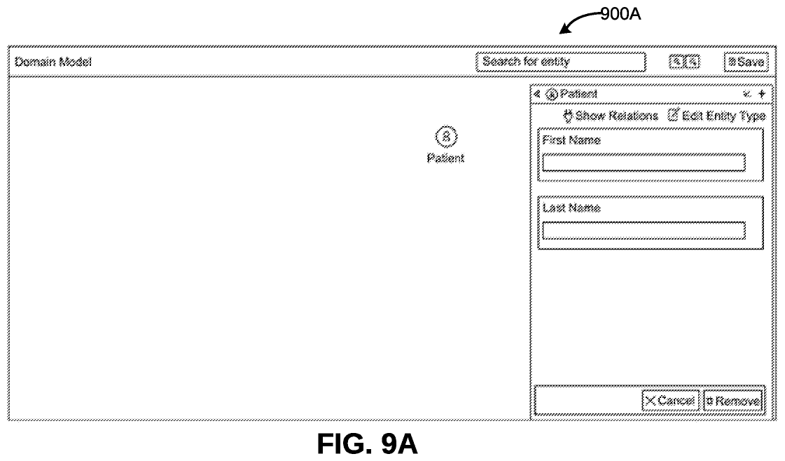

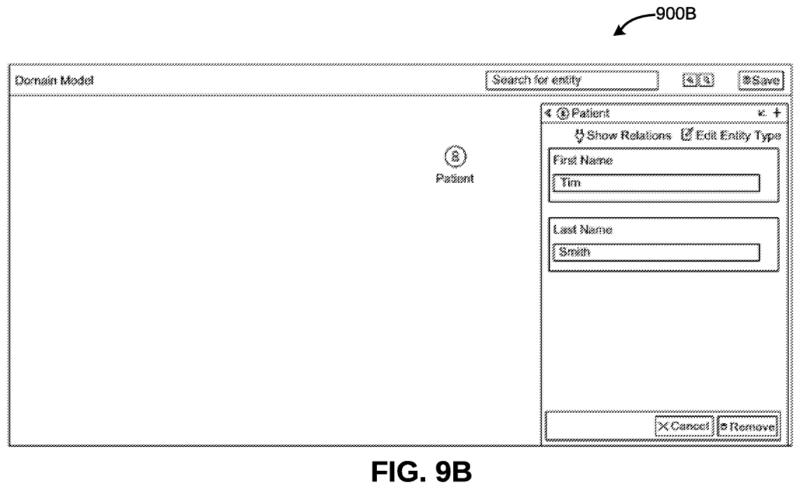

Referring now to FIGS. 9A-9B, UIs 900A and 900B for inputting information related to the entity attributes are illustrated, in accordance with an embodiment of the present invention. After creating and associating attributes to the entity ‘patient’ 702 via the UI 800 in the FIG. 8, the information related to the entity attributes may be inputted, using the UIs 900A and 900B as shown in the FIGS. 9A and 9B. At the UIs 900A and 900B, the information for the present use case is captured. Such as, for the ‘first name’ and ‘last name’ of the patient, information ‘Tim’ and ‘Smith’ are inputted and captured. Thus, the attributes ‘first name’ and ‘last name’ of the entity ‘patient’ contains the information ‘Tim’ and ‘Smith’ respectively.

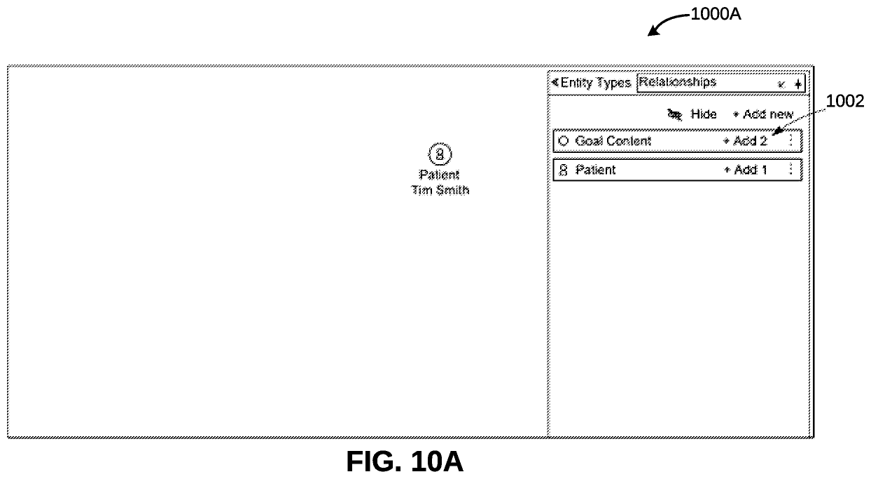

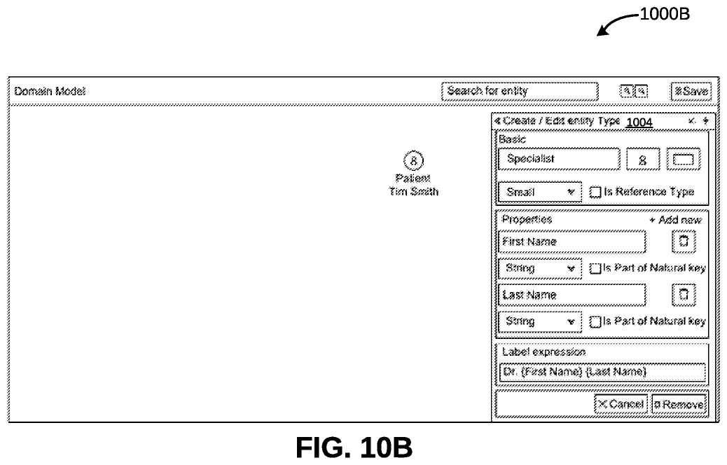

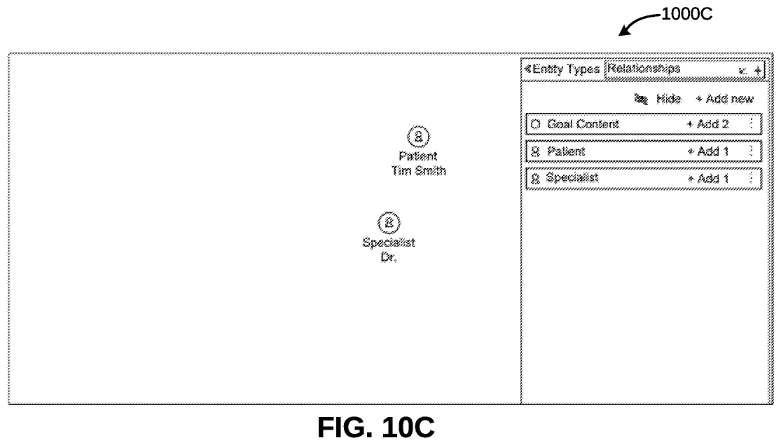

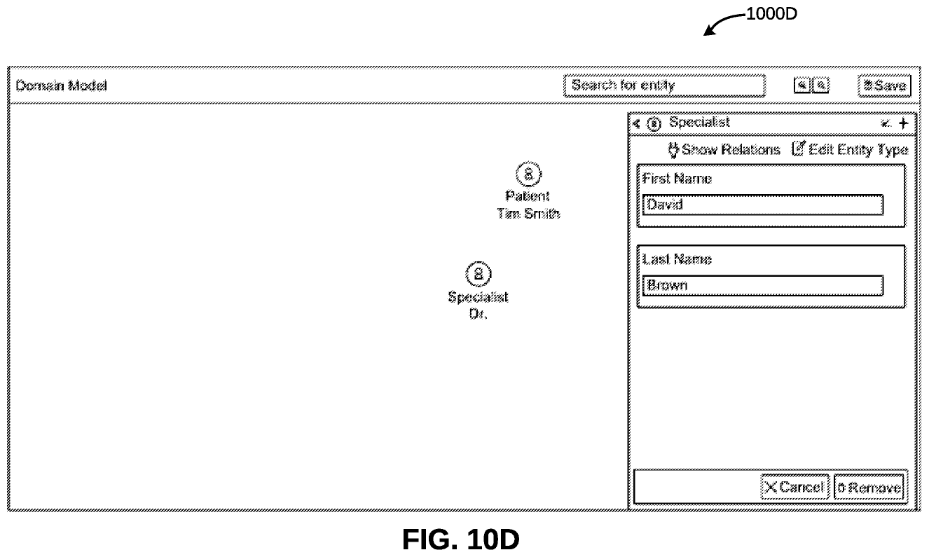

Referring now to FIGS. 10A-10D, UIs 1000A-1000D for adding, creating and representing another entity are illustrated, in accordance with an embodiment of the present invention. In the FIGS. 10A-10D, the UIs 1000A-1000D, show adding, creating, and representing another entity called ‘specialist’. Also, the UIs 1000A-1000D show adding and creating attributes for the entity ‘specialist’, along with entering and capturing information for the attributes of the entity ‘specialist’. Thus, the UIs shown in the FIGS. 6-13 may be employed in creating, adding, modifying and representing multiple entities, while also adding and creating attributes for the multiple entities, and thereafter entering and capturing information for the attributes of the multiple entities to be represented in the multi-dimensional data model.

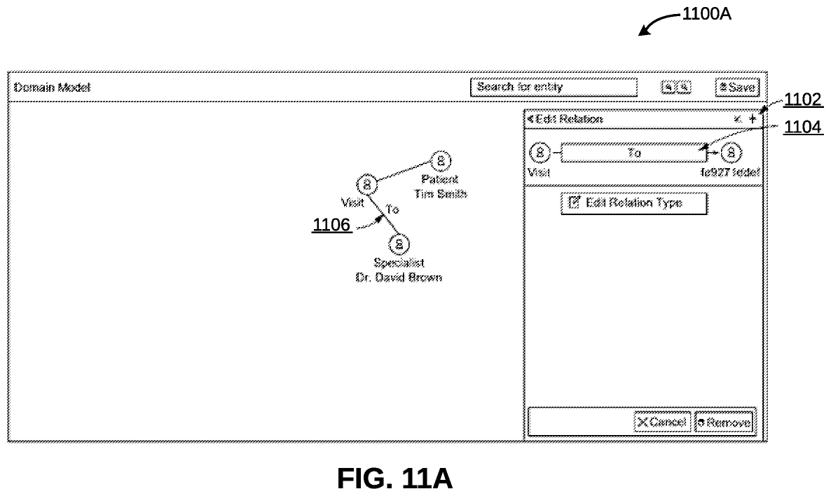

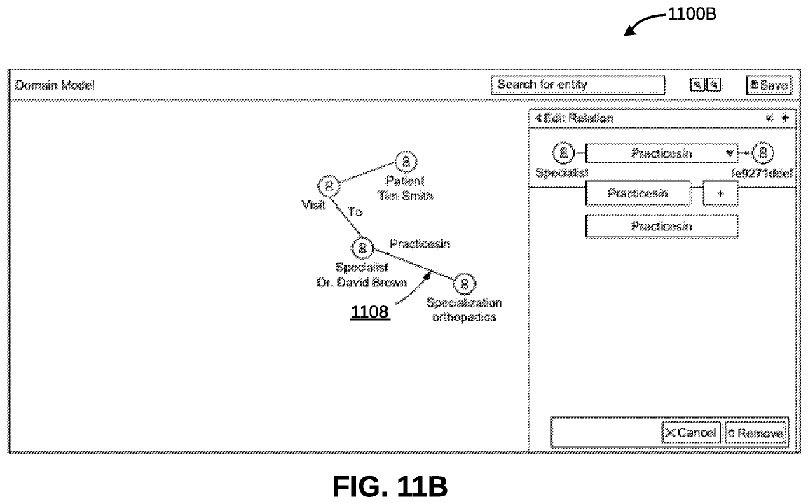

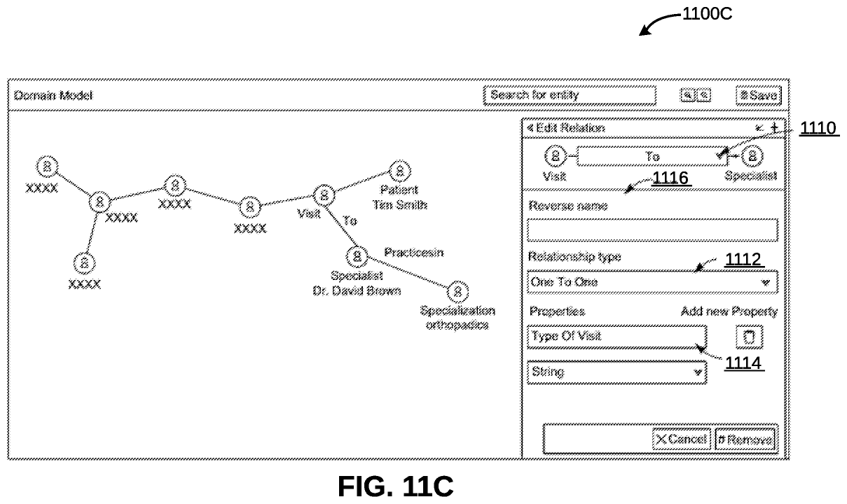

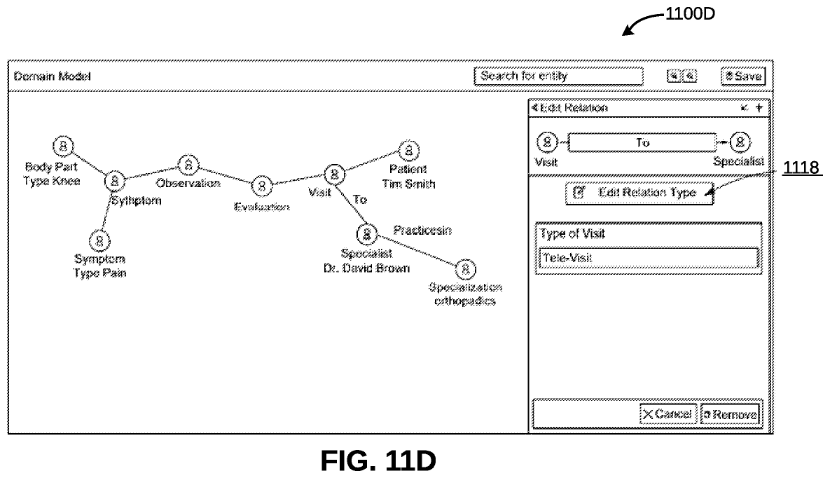

Referring to FIG. 11A-11B, UIs 1100A-1100B employed to create relationship types between data entities is illustrated, in accordance with an embodiment of the present invention. As an example, as shown in the UI 1100A, a tab ‘1102’ in the creation section may be used to create, add and modify relationship types between the entities. A tab ‘1104’ may be used to provide attributes to the created relationship type. After creating a relationship type between the entity ‘visit’ and ‘specialist’, and providing attributes, such as a label ‘to’ to the created relationship type, the relationship type 1106 is created and represented in the data model, in the model representation section of the UI 1100A.

Similarly, the model creation device 102 may create and represent more relationship types between the entities. For example, the UI 1100B in the FIG. 11B, creates and represents a relationship type ‘practicesin’ 1108 between the entities ‘specialist’ and ‘specialization’. Additionally, the relationship types may be unidirectional and/or bidirectional.

Referring to FIG. 11C, a UI 11000 employed to provide and associate attributes to relationship types is illustrated, in accordance with an embodiment of the present invention. After creating the relationship types, the model creation device 102 may also provide one or more attributes or properties to the relationship types, and may further help in inputting and capturing information in the created attributes. For example, as shown in the FIG. 11C, the UI 11000 may be employed to provide and associate one or more attributes or properties to the relationship types. The creation section of the UI 11000 may have one or more tabs to provide attributes to the relationship types, such as tabs 1110, 1112 and 1114. As also may be seen, using a tab 1116, a reverse name may also be provided to the relationship type. Thus, the relationship types may be unidirectional and/or bi-directional.

Referring to FIG. 11D, a UI 1100D employed for editing is illustrated, in accordance with an embodiment of the present invention. Further, the model creation device 102 may also help in editing the relationship attributes created and/or information captured in the attributes. For example, it may be seen in the UI 1100D in the FIG. 11D, a tab 1118 may be used to edit the relationship attributes and/or information captured in the attributes.

Therefore, in the UIs 1100A-1100D, the model creation device 102 may create relationship types between multiple entities, provide and associate one or more relationship attributes to them, and capture information in the attributes created for the relationship types. Thus, capturing knowledge in the relationship types between the multiple entities.

This enhances the relational data model supporting graph-based RDBMS, wherein not only relationships are added, but multi-dimensional relationships between multiple entities may be added, and attributes, information and knowledge may further be captured for these attributes associated with the entities and the relationships as well.

As described and shown in the exemplary situation in the UIs 600-1100D in the FIGS. 6-11D, multiple entities and the relationships between them may represent a use case. Further, it can also be seen that the model creation device 102 may efficiently employ this use case to create a graph-based relational data model from the scratch. Similarly, more entities and relationships may be created and modelled for the same use case, which elaborates the use case more, or entities and relationships may be created and modelled for different use cases, which enhances the multi-dimensional relational data model further by capturing more and more information for multiple use cases.

In an embodiment, different use cases may be related to each other, and this relationship may be represented by the graph based UIs 600-1100D. By using multiple use cases, the present model creation device 102 may create and model a data model, even in the schema less nature of graph based data models/databases.

As and when more and more entities and relationship types are created and modelled using the model creation device 102, more and more entities and relationship types are added in the creation section of the UI, while also represented in the model representation section of the UI.

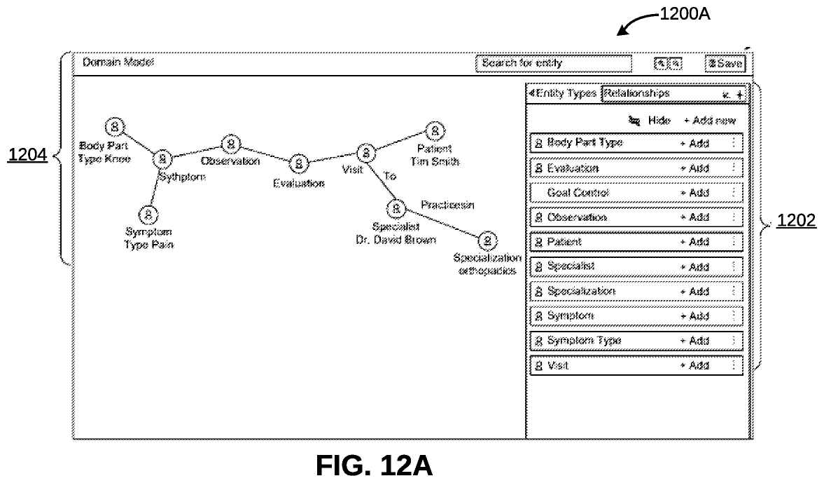

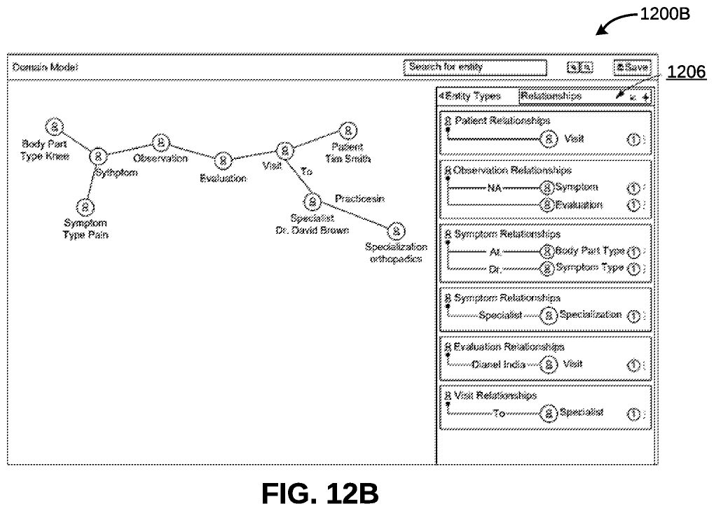

Referring now to FIGS. 12A-12B, directed graph-based UIs 1200A-1200B that represent a multi-dimensional data model are illustrated, in accordance with an embodiment of the present invention. For example, the UI 1200A of the FIG. 12A, lists and shows multiple entities and relationship types (1202) which are created in the creation section of the UI. And, the created multiple entities and relationship types are being represented, in the multi-dimensional data model (1204), in the model representation section of the UI.

Furthermore, the UI 1200B in the FIG. 12B may be used to expand the multiple entities and relationship types for showing the information and the relationships between multiple entities. A tab 1206 may be used to expand the entity types further, which shows how multiple entities are related to each other.

Thus, at least implementing and employing the graph-based UIs shown in the FIGS. 6-12, the model creation device 102 may help in data modelling in the most natural way, reflecting and representing closely an actual domain model. The data model created by the present model creation device 102 may represent more closely to a verbal language which may be naturally used to describe a use case or an example, for example the English language which may be used by an expert to describe use cases. The model creation device 102 may capture the information in the most natural language sentence, while capturing the most basic or minute information as well. Furthermore, the model creation device 102 may capture real life data.

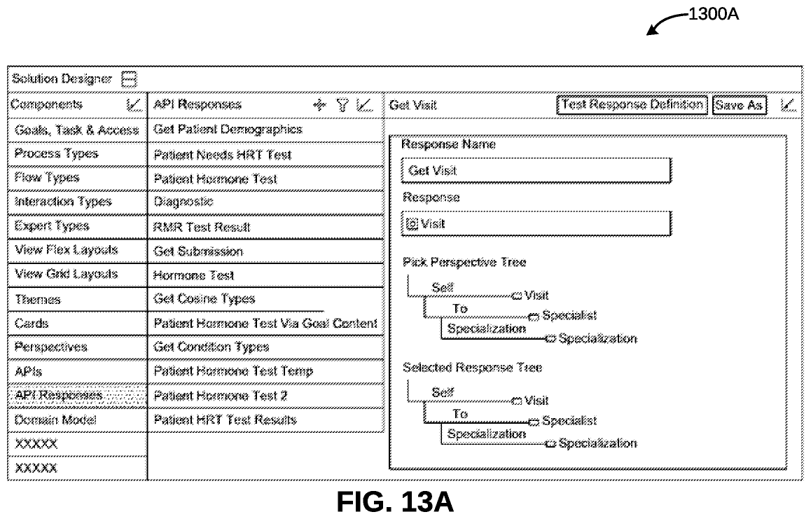

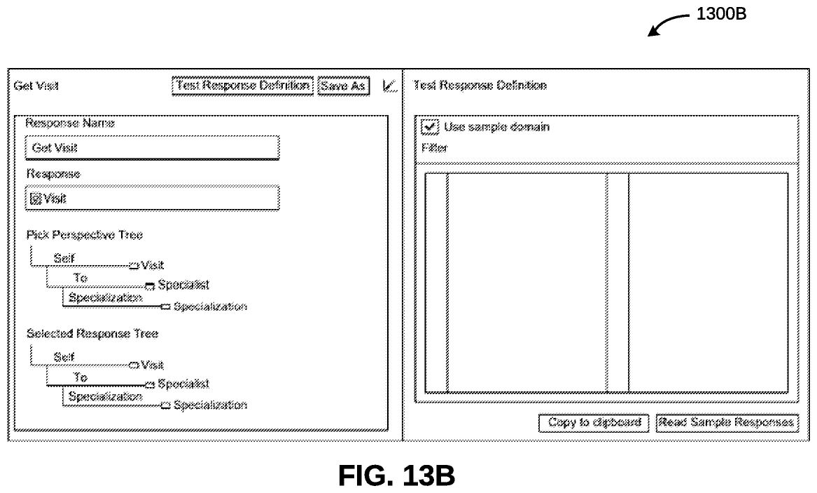

Referring to FIGS. 13A and 13B, the user interfaces 1300A and 1300B where the data captured in the multi-dimensional data model may be used as sample data for various applications are illustrated, in accordance with some embodiments. The UIs 1300A and 1300B may show how the created data model, in the FIGS. 6-12, may be automatically used as sample data for various applications such as helps in using the data as mock data to build rest of software application. Thus, the model creation device 102 may help in creating rich data model.

Advantageously, the present disclosure provides an intuitive tool to model a domain naturally and very closely to actual domain model, and has the ability to model data without loss of fidelity. Also, the present disclosure provides an intuitive tool to create sample data, during the process of data modeling that implements use cases approach, and this sample data also helps in using the data as mock data to build rest of software application, for e.g. whether to build screens that connect to mock data to realize data visualization or to build APIs that are defined to fetch appropriate data. Thus, mock data is created even at the time of envisioning the data model in the present invention.

Advantageously, the present system 100 may have at least the following applications and advantages.

Identify and document domain's entities, relationships and their attributes through example use cases. The present system 100 may define and enhance the domain's data model incrementally from gradually unfolding data storage/consumption needs as it traverses use case/process examples.

Makes the data modelling process a lot easier by bringing the process closer to the way human's think about the domain, it's use cases/processes, one at a time.

The example driven process not only makes it easier to model by reducing the scope of the domain that is in focus at any given time, it also reduces any misses and rework.

By covering most of the important use cases/processes through examples, the present system 100 may get to the design of a more complete data model.

The example data that is created along the way doubles up to serve other really important needs, such as: a) describing the data model in a natural way (by example) to those that need to understand it at a later time, b) For use as test data for testing software components (APIs, Cards etc.) during development.

Further, the present system 100 may also provide a graph based user interface to define, describe, document and maintain them, because of at least the following advantages:
a) Data Model Graph UI let users create property graph database model, a simple yet expressive tool for representing connected data. Property graphs capture complex domains in an expressive and flexible fashion, while the underlying graph database makes it easy to develop applications that manipulate our graph models.
B) Easy to use visual modelling tool for defining and managing at least the following:
   1. Entity Types (name and attributes, descriptions for entity type and it's each attributes, visual descriptive elements: color, icon, label, search)
   2. Relationship Types (name and their attributes, descriptions of relationship type and it's each attribute, direction, name of relationship in reverse, relationship type cardinality—one-to-one, one-to-many, many-to-one, many-to-many)
   3. Entities with relationships representing data capture and consumption needs of example use cases/processes (Entities with Relationships are instances of entity types and relationship types from example use cases)
   4. Ability to move and rearrange the entities/relationships on the design canvas.
   5. Ability to search for entities on the design canvas.
   6. Ability to view a sub-graph as well as save it as a named view for future quick access to that sub-graph.
   7. Ability to view the number of entities of each entity type that are on the graph to help us identify any unused entity types that may no longer be relevant and can be deleted.
   8. Ability to “Show All” & “Hide All”
   9. Ability to “Show” or “Hide” an Entity Type
   10. Ability to view all relationships from a given entity type for all entity types.
   11. Ability to view the number of relationships of each relationship type that are on the graph to help identify any unused relationship types that may no longer be relevant and can be deleted.
   12. Ability to zoom in/out on the design canvas using the buttons as well as touch (pinch zoom).
   13. On the Roadmap: a graphical view of only the data model (not the example data)—that is entity types & relationship types only to provide the abstract view that some users would like to have.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A method of creating a data model, the method comprising:
receiving, via a first User-interface, an input to create a process entity, wherein the process entity is configured to act as first-class citizen;
creating, via a second User-Interface, a node corresponding to the process entity to further generate a nodal network, wherein the second User-Interface comprises a plurality of nodes;
dynamically receiving, via the first User-interface, one or more attributes associated with a selected entity;
annotating, via the second User-interface, a node corresponding to the selected entity based on the one or more attributes received; and
dynamically creating, via the second User-Interface, a link between a first node and a second node in the nodal network, wherein creating the link comprises:
receiving, via the second User-Interface, a selection of the first node;
receiving, via the second User-Interface, a selection of the second node;
generating the link between the first node and the second node; and
receiving, via the second User-Interface, a direction input associated with the link; and
defining the direction of the link between the first node and the second node,
wherein the link is configured to act as a first-class citizen.

2. The method of claim 1, wherein:
the nodal network is rendered in a vector format via the second User-Interface, and
the nodal network is configured to be zoomed-in and zoomed-out.

3. The method of claim 1, further comprising:
defining one or more attributes to the link between the first node and the second node in the nodal network, wherein the link between the first node and the second node in the nodal network is classified in one or more categories based on the one or more attributes.

4. The method of claim 1, further comprising:
selecting one or more aesthetic attributes for each node in the nodal network, wherein the one or more aesthetic attributes comprise: a shape, a size, and a colour.

5. The method of claim 1, further comprising:
receiving at least one of:
a selection of a portion of the one the nodal network, via the second User-Interface;
a selection of an attribute from the one or more attributes associated with a node; or
a selection of an attribute from the one or more attributes associated with a link; and
rendering a portion of the nodal network based on one of the selection of the portion of the one the nodal network, the selection of the attribute from the one or more attributes associated with the node, or the selection of the attribute from the one or more attributes associated with the link.

6. A system for creating a data model, the system comprising:
a processor; and
a memory communicatively coupled to the processor, wherein the memory stores processor-executable instructions, which, on execution, cause the processor to:
receive, via a first User-interface, an input to create a process entity, wherein the process entity is configured to act as first-class citizen;
create, via a second User-Interface, a node corresponding to the process entity to further generate a nodal network, wherein the second User-Interface comprises a plurality of nodes;
dynamically receive, via the first User-interface, one or more attributes associated with a selected entity;
annotate, via the second User-interface, a node corresponding to the selected entity based on the one or more attributes received; and
dynamically create, via the second User-Interface, a link between a first node and a second node in the nodal network, wherein creating the link comprises:
receive, via the second User-Interface, a selection of the first node;
receive, via the second User-Interface, a selection of the second node;
generate the link between the first node and the second node; and
receive, via the second User-Interface, a direction input associated with the link; and
define the direction of the link between the first node and the second node,
wherein the link is configured to act as a first-class citizen.

7. The system of claim 6, wherein:
the nodal network is rendered in a vector format via the second User-Interface, and
the nodal network is configured to be zoomed-in and zoomed-out.

8. The system of claim 6, wherein the processor-executable instructions further cause the processor to define one or more attributes to the link between the first node and the second node in the nodal network, wherein the link between the first node and the second node in the nodal network is classified in one or more categories based on the one or more attributes.

9. The system of claim 6, wherein the processor-executable instructions further cause the processor to select one or more aesthetic attributes for each node in the nodal network, wherein the one or more aesthetic attributes comprise: a shape, a size, and a colour.

10. The system of claim 6, wherein the processor-executable instructions further cause the processor to:
receive at least one of:
a selection of a portion of the one the nodal network, via the second User-Interface;
a selection of an attribute from the one or more attributes associated with a node; or
a selection of an attribute from the one or more attributes associated with a link; and
render a portion of the nodal network based on one of the selection of the portion of the one the nodal network, the selection of the attribute from the one or more attributes associated with the node, or the selection of the attribute from the one or more attributes associated with the link.

11. A non-transitory computer-readable medium storing computer-executable instructions for creating a data model, the computer-executable instructions configured for:
receiving an input to create a process entity via a first User-Interface, wherein the process entity is configured to act as first-class citizen;
creating a node corresponding to the process entity to further generate a nodal network via a second User-Interface, wherein the second User-Interface comprises a plurality of nodes;
dynamically receiving one or more attributes associated with a selected entity via the first User-Interface;
annotating a node corresponding to the selected entity based on the one or more attributes received via the second User-Interface; and
dynamically creating a link between a first node and a second node in the nodal network via the second User-Interface, wherein creating the link comprises:
receiving a selection of the first node;
receiving a selection of the second node;
generating the link between the first node and the second node; and
receiving a direction input associated with the link; and
defining the direction of the link between the first node and the second node,
wherein the link is configured to act as a first-class citizen.

12. The non-transitory computer-readable medium of the claim 11, wherein:
the nodal network is rendered in a vector format via the second User-Interface, and
the nodal network is configured to be zoomed-in and zoomed-out.

13. The non-transitory computer-readable medium of the claim 11, wherein the computer-executable instructions further configured for defining one or more attributes to the link between the first node and the second node in the nodal network, wherein the link between the first node and the second node in the nodal network is classified in one or more categories based on the one or more attributes.

14. The non-transitory computer-readable medium of the claim 11, wherein the computer-executable instructions further configured for selecting one or more aesthetic attributes for each node in the nodal network, wherein the one or more aesthetic attributes comprise: a shape, a size, and a colour.

15. The non-transitory computer-readable medium of the claim 11, wherein the computer-executable instructions further configured for:
receiving at least one of:
a selection of a portion of the one the nodal network, via the second User-Interface;
a selection of an attribute from the one or more attributes associated with a node; or
a selection of an attribute from the one or more attributes associated with a link; and
rendering a portion of the nodal network based on one of the selection of the portion of the one the nodal network, the selection of the attribute from the one or more attributes associated with the node, or the selection of the attribute from the one or more attributes associated with the link.
